(19) United States
(12) Patent Application Publication
(10) Pub. No.: US 2004/0120979 A1
(43) Pub. Date: Jun. 24, 2004

(54) Delivery systems comprising biocompatible and bioerodable membranes

(76) Inventors: Blake J. Roessler, Ann Arbor, MI (US); James R. Baker JR., Ann Arbor, MI (US); Anna U. Bielinska, Ypsilanti, MI (US)

Correspondence Address: MEDLEN &CARROLL, LLP, Suite 2200, 220 Montgomery Street, San Francisco, CA, 94104, US

(21) Appl. No.: 09/867,924

(22) Filed: May 30, 2001

Related U.S. Application Data
(60) Provisional application No. 60/208,728, filed on Jun. 2, 2000.

(51) Int. Cl. C08G 63/91; C08G 63/48; A61F 2/00

(52) U.S. Cl. 424/426; 525/54.1

(57) ABSTRACT

The present invention relates to novel compositions and methods for delivering substances to target tissues and cells by contacting the targets with delivery systems associated with membranes (e.g., biocompatible or bioerodable membranes). More particularly, the present invention is directed to dendrimer-based methods and compositions for use in disease therapies, wound healing, and generally, improved gene transfection and compound delivery to target cells and tissues in vitro and in vivo.

This Application claims priority to Provisional Application 60/208,728 filed June 2, 2000.

This invention was made in part during work partially supported by the U.S. government under NIH NO1-AR-6-2226. The government has certain rights in the invention.

### FIELD OF THE INVENTION

The present invention relates to novel compositions and methods for delivering substances to target tissues and cells by contacting the targets with delivery systems associated with membranes (e.g., biocompatible or bioerodable membranes). More particularly, the present invention is directed to dendrimer-based methods and compositions for use in disease therapies, wound healing, and generally, improved gene transfection and compound delivery to target cells and tissues in vitro and in vivo.

### BACKGROUND OF THE INVENTION

The primary goal in the wound treatment is to achieve wound closure. Open cutaneous wounds represent one major category of wounds and include burn wounds, neuropathic ulcers, pressure sores, venous stasis ulcers, and diabetic ulcers. Numerous factors can affect wound healing, including malnutrition, infection, pharmacological agents (e.g., actinomycin and steroids), diabetes, advanced age, and endogenous factors. Another factor affecting wound healing is the extent of the damage to the underlying vascular tissues. Large wounds with substantially compromised vascularization (e.g., microvascular disease) often do not heal properly because oxygen cannot be supplied to the wound in sufficient quantities. Moreover, certain types of chronic wounds (e.g., diabetic ulcers, pressure sores) and the wounds of certain subjects (e.g., recipients of exogenous corticosteroids) are also problematic to treat.

Advances in wound healing have generally been realized. For instance, the most commonly used conventional methods to assist in wound healing involves the use of wound dressings. In the 1960s, a major breakthrough in wound care occurred when it was discovered that wound healing with a moist occlusive dressings was, generally speaking, more effective than the use of dry, non-occlusive dressings (Winter, Nature 193:293 [1962]). Today, numerous occlusive type dressings are routinely used, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (nonwoven composites of fibers from calcium alginate), and cellophane (cellulose with a plasticizer) [Kannon and Garrett, Dermatol. Surg. 21:583 (1995); Davies, Burns 10:94 (1983)].

Additionally, several pharmaceutical methods have been utilized in an attempt to improve wound healing. For example, treatment regimens involving zinc sulfate have been utilized by some practitioners. The efficacy of these regimens has been primarily attributed to their reversal of the effects of sub-normal serum zinc levels (e.g., decreased host resistance and altered intracellular bactericidal activity) [Riley, Am. Fam. Physician 24:107 (1981)]. While other vitamin and mineral deficiencies have also been associated with wound healing (e.g., deficiencies of vitamins A, C and D; and calcium, magnesium, copper, and iron), there is no strong evidence that increasing the serum levels of these substances above their normal levels actually enhances wound healing.

However, for those suffering from many of the problematic wounds mentioned above, even occlusive dressings and the various pharmaceutical methods mentioned have provided little amelioration for their suffering. Thus, except in limited circumstances, the promotion of wound healing with these agents has met with varied success. What is needed are safe and effective compositions and methods for delivering wound healing therapeutic substances and factors to target tissues and cells by contacting the target with effective delivery systems in vitro and in vivo.

### SUMMARY OF THE INVENTION

The present invention relates to novel compositions and methods for delivering substances (e.g., therapeutic substances) to target tissues and cells by contacting the targets with delivery systems associated with membranes (e.g., biocompatible or bioerodable membranes). More particularly, the present invention is directed to dendrimer-based methods and compositions for use in disease therapies, wound healing, and generally, improved gene transfection and compound delivery to target cells and tissues in vitro and in vivo.

For example, the present invention provides compositions comprising a membrane associated with at least one dendrimer, said dendrimer comprising at least one biological agent. The present invention is not limited by the nature of the membrane. In some embodiments, the compositions of the present invention is in contact with a biological tissue (e.g., a tissue of a host in vivo and a cultured tissue in vitro). In preferred embodiments of the present invention, the membrane comprises a biocompatible membrane (e.g., a biocompatible membrane in contact with a tissue). Any type of biocompatible membrane is contemplate including, but not limited to, PLGA membranes and collagen membranes. In certain embodiments where a collagen membrane is utilized the composition may further comprise a collagenase. In yet other preferred embodiments, the membrane comprises a bioerodable membrane. The bioerodable membrane may comprise a single bioerodable layer or multiple bioerodable layers (e.g., multiple bioerodable layers, each with a distinct biological agent associated with it). In some embodiments, the membrane is desiccated.

In some embodiments of the present invention, the dendrimer is covalently attached to the membrane. For example, in some embodiments, the dendrimer is attached to a surface of the membrane (e.g., attached so that it is exposed to the environment). In other embodiments, the dendrimer is encompassed within the membrane (e.g., within a bioerodable membrane such that it is not exposed to the environment until at least partial degradation of the bioerodable membrane). In some embodiments, the membrane is associated with a plurality of dendrimers. For example, the membrane may be attached at a plurality of dendrimers each comprising a different agents.

In some embodiments of the present invention, the agent is attached to a surface of the dendrimer (e.g., attached so that it is exposed to the environment). In other embodiments, the dendrimer is encompassed within the dendrimer (e.g., within the interior of the dendrimer such that it is not directly exposed to the environment). The present invention is not limited by the nature of the biological agent. In some preferred embodiments, the agent comprises a therapeutic agent. In particularly preferred embodiments, the therapeutic agent comprises nucleic acid (e.g., DNA, RNA, antisense oligonucleotides). Where the agent is DNA, the present invention is not limited by the nature of the DNA. In certain preferred embodiments, the DNA comprises a gene encoding a protein that promotes wound healing (e.g. a growth factor). In other preferred embodiments, the DNA comprises a gene encoding a protein that promotes tissue vascularization (e.g., a growth factor). In other embodiments, the therapeutic agent comprises a protein (e.g., a protein that promotes wound healing or tissue vascularization).

The present invention also provides a method comprising providing 1) a tissue and 2) a composition comprising a membrane associated with at least one dendrimer, said dendrimer comprising at least one biological agent; and contacting the tissue with the composition. Any of the compositions described above find use with the methods. The present invention is not limited by the nature of the tissue. For example, in some embodiments, the tissue comprises cultured cells in vitro. In some embodiments, the tissue comprises ex vivo tissue obtained from a subject. In still other embodiments, the tissue comprises tissue of a subject (e.g., skin, organ, or other tissue in vivo). In preferred embodiments, the tissue comprises skin cells. In some embodiments, the step of contacting the composition with the tissue comprises placing a composition on a wound of a subject. In other embodiments, the contacting comprises placing the composition on a lesion of the subject.

The present invention also provides a desiccated membrane capable of transfecting a tissue (e.g., capable of incorporating a nucleic acid into a tissue). In some embodiments, the membrane comprises at least one dendrimer. In preferred embodiments, the dendrimer comprises at least one biological agent (e.g., nucleic acid). In preferred embodiments, the tissue comprises skin tissue. The present invention further provides a method comprising providing 1) a tissue and 2) a composition comprising a desiccated membrane capable of transfecting said tissue; and contacting the tissue with the composition.

### DESCRIPTION OF THE FIGURES

The following figures form part of the specification and are included to further demonstrate certain aspects and embodiments of the present invention. The invention may be better understood by reference to one or more of these figures in combination with the detailed description of specific embodiments presented herein.

FIG. 1 shows a graph demonstrating that cell cultures, when incubated with the compositions and methods of the present invention, express transgene.

FIG. 2A shows a graph demonstrating the effects on transfection with certain dendrimer/DNA charge ratios. FIG. 2B shows a graph demonstrating a time course of expression for cells contacted with the methods and compositions of the present invention.

FIG. 3 shows a graph showing the effects on expression of exposing collagen membranes to collagenase.

FIG. 4A shows a graph depicting the effects of phosphatidylglycerol on the in situ transfection. FIG. 4B also depicts the effects of phosphatidylglycerol on the in situ transfection. FIG. 4C provides an image showing transgene expression in primary human keratinocytes (PHEK) contacted with the compositions and methods of the present invention.

FIG. 5 shows a graph illustrating the effects of varying dendrimer/DNA charge ratios on transfection efficiency.

### GENERAL DESCRIPTION OF THE INVENTION

The present invention relates to novel compositions and methods for delivering substances (e.g., therapeutic substances) to target tissues and cells by contacting the targets with delivery systems associated with membranes (e.g., biocompatible or bioerodable membranes). More particularly, the present invention is directed to dendrimer-based methods and compositions for use in disease therapies, wound healing, and generally, improved gene transfection and compound delivery to target cells and tissues in vitro and in vivo.

Advances in gene therapy technology have extended the classic concept of drug delivery toward the inclusion of nucleic acids as therapeutic agents (See e.g., Gerwitz et al., Blood 92:712 [1998]; Ye et al, Mol Med Today 4:431 [1998]). Therapeutic genes, antisense oligonucleotides, or ribosomes can be delivered, with varying levels of effectiveness, to target cells by various viral and non-viral delivery systems. The present invention demonstrates that dendrimer-based systems have many advantages over other methods and compositions for delivering nucleic acids, proteins, and other factors (e.g., drugs) of interest to host cells. For example, the present invention provides dendrimer-based delivery systems that comprise dendrimer complexes and one or more biologically active or therapeutic agents for wound healing, transfection, and general delivery of proteins and therapeutics to target cells or tissues. In some embodiments of the present invention, cationic (polyamidoamine) PAMAM dendrimers are used as synthetic carriers of nucleic acids and other therapeutics. PAMAM dendrimers are spherical, nanoscopic polymers with a molecular architecture characterized by the regular dendritic branching and radial symmetry (See e.g., Tomalia et al, Agnew Chem Int Ed Engl 29:138 [1990]; Frechet, Science 263:1710 [1994]). Positive charge density due to the presence of protonized primary amine groups on the surface enables these molecules to form electrostatic complexes with polyanionic biological macromolecules including various forms of nucleic acids. Dendrimers are highly efficient for in vitro transfection and appear to be non-cytotoxic in the concentrations suitable for gene transfer (See e.g., Kukowska-Latallo et al., Proc Natl Acad Sci USA 93:4897 [1996]; Bielinska et al., Nucleic Acids res 24:2176 [1996]). Studies suggests that these polymers are not immunogenic or carcinogenic, enhancing their potential as in vivo gene transfer systems (See e.g., Roberts et al., J Biomed Mater res 30:53 [1996]). However, the present invention is not limited by the nature of the dendrimers. Indeed, dendrimers suitable for use with the present invention include, but are not limited to, polyamidoamine (PAMAM), polypropylamine (POPAM), polyethylenimine, poly(propylene imine), iptycene, aliphatic poly(ether), and/or aromatic polyether dendrimers.

In some embodiments of the present invention, the dendrimer complexes degrade in a time dependent manner under physiological conditions (e.g., to provide time release delivery of an agent or agents). In other embodiments, the dendrimer complexes resist degradation for a period of time under physiological conditions, and then proceed to degrade.

In other embodiments, degradation of the dendrimer complexes is influenced by the surface chemistries of the dendrimers utilized. Thus, particular dendrimer complexes may be selected or designed that degrade under particular physiological conditions or under an exogenous cue provided either at administration, or at a selected biological event after administration.

In still further embodiments, the dendrimer complexes of the present invention may comprise one or more layers of dendrimer structure such that one or more layers (i.e., concentric layers) have associated therewith, one or more biologically active or therapeutic agents. The biologically active or therapeutic agents sequestered in these dendrimer complexes may comprise one or more particular biologically active or therapeutic agents. Thus, the present invention also contemplates that, where one or more one biologically active or therapeutic agents are associated with a dendrimer complex, these compounds may be similar throughout the various portions (e.g., layers) of a particular dendrimer complex. Alternatively, one or more dissimilar biologically active or therapeutic agents may be associated with dendrimer complexes per layer. The present invention is not limited by the particular biologically active or therapeutic agents associated with the dendrimer complexes, moreover, each biologically active or therapeutic agent may further comprise pharmaceutically accepted compounds (e.g., one or more excipients, adjutants, diluents, etc.).

In some embodiments, one or more dendrimer complexed with one or more associated biologically active or therapeutic agents may be further associated with one or more biocompatible or bioerodable membranes. In some embodiments of the present invention, the dendrimer complexes are associated with solid or semi-solid biocompatible or bioerodable membranes. The present invention contemplates that suitable biocompatible and bioerodable membranes may comprise sheets, foams, viscous layers, gelatins, or mucous like preparations.

In some embodiments, the present invention provides methods where dendrimer complexes associated with one or more biocompatible or bioerodable membranes are administered sequentially or substantially sequentially to target cells and/or tissues. In still further embodiments, the present invention provides methods and compositions wherein one or more dendrimer complexes associated with one or more biocompatible or bioerodable membranes are administered simultaneously or substantially simultaneously to a cell or tissue (e.g., in vitro or in vivo).

In some embodiments, the biocompatible or bioerodable membranes may be selected to have substantial permeability to gases, anabolites, metabolites, organic and inorganic macromolecules, factors, cofactors, coenzymes, and the like. In other embodiments, the biocompatible or bioerodable membrane selected are substantially impermeable to such factors.

In some embodiments, the biocompatible or bioerodable membrane is beneficially associated with anabolites, antibiotics, factors, cofactors, coenzymes, proteins, etc. associated with promoting wound healing and or tissue vascularization. The present invention also provide biocompatible or bioerodable membrane that are substantially bacteria, fungi, mycoplasma, and pyrogen free.

In some embodiments, the dendrimers are associated with the surface of biocompatable or bioerodable membrane such that contacting the membrane with a cell or tissue results in direct exposure of the dendrimer complexes to the cell or tissue to be treated. In other embodiments, the biocompatable or bioerodable membrane with associated dendrimer complexes are contacted to a region of a host distal from the region to be treated. Thus, in 1.5 some embodiments, the dendrimer-based complexes contemplate the systemic delivery of biologically active or therapeutic agents.

In some embodiments of the present invention, the dendrimers-based complexes of the present invention are associated with surgical or wound cover adhesives, biodegradable surgical sutures, or packaging containers (e.g., polyurethanes, urethane acrylates, combined polyurethanes, and the like). In other embodiments, the dendrimer-based complexes of the present invention are associated with dermal substitutes, or guided tissue regeneration compositions, and the like.

In still other embodiments, the surface chemistries of the biocompatible or bioerodable membranes are altered or selected such that the dendrimer complexes of the present invention disassociate from the supporting membranes. In some embodiments, the disassociation of the dendrimer complexes is controlled to yield a dispersion of the complexes over a therapeutic time period. In other embodiments, the disassociation of the dendrimer complexes is controlled so that the disassociation is cued to an endogenous physiological event (e.g., exposure to acidic pH, cleavage enzymes, hydrolytic enzymes, ligands, etc.). In still further embodiments the disassociation is cued to an exogenous physiological event (e.g., exposure to light, heat, a second chemical modality, etc.). In some embodiments, the dissociation of dendrimer complexes from the biocompatable or bioerodable membranes is actuated by endogenous or exogenous agents (e.g., lytic or hydrolytic enzymes, or inorganic agents).

In some embodiments, the dendrimers complexes are employed to topically deliver biologically active or therapeutic agents. In some embodiments, where topical administration of therapeutics to epidermal keratinocytes is desired, a carrier that enables prolonged contact is provided, enhancing skin permeability and extending delivery (See e.g., Choate et al., Human Gene Ther 8:1659 [1997]; Trainer et al., Human Mol Gen 6:1761 [1997]; Jain et al., Drug Dev Ind Pharm 24:703 [1998]). Various biocompatible systems have been tested for their feasibility to serve as platforms for delivering traditional pharmaceuticals including various hormones, nicotine, antihypertensives, etc. (See e.g., Luck et al., J. Control Rel 55:107 [1998]; Webber et al., J. Biomed Mater Res 41:18 [1998]; Garcia-Contreras et al., Pharm Dev Tech 2:53 [1997]). In some embodiments of the present invention, the dendrimer-based complexes are employed to delivery nucleic acid and therapeutic agents to mucosal cells and tissues (e.g., alveolar, buccal, lingual, masticatory, or nasal mucosa, and other tissues and cells which line hollow organs or body cavities). In particular embodiments, the dendrimer-based delivery systems of the present invention are employed to deliver biologically active or therapeutic agents to wounds of the hosts's integument, or to internal lesions.

In some embodiments, the biologically active or therapeutical agents are associated with the dendrimer-based delivery systems of the present invention by association as a surface coating on a suitable biocompatible or bioerodable membrane. In other embodiments, the biologically active or therapeutical agents are associated with the dendrimer-based delivery systems of the present invention by incorporation into a suitable biocompatible or bioerodable membrane. In other embodiments, the biologically active or therapeutical agents are associated directly onto or into the dendrimer-based complexes of the present invention.

In some embodiments, the biologically active or therapeutic agents of the present dendrimer-based delivery systems comprise nucleic acid sequences. In certain embodiments directed to wound healing, the dendrimer-based delivery systems comprise nucleic acid sequences encoding cellular mediators, growth factors, and biologically active and therapeutic agents associated with wound healing. In other embodiments, one or more of the aforementioned agents, or other agents, are associated with the dendrimer-based deliver systems of the present invention.

In embodiments directed to promote wound healing, the biologically active or therapeutic agent components of the dendrimer-based delivery systems comprise agents that promote one or more of the three stages of wound healing. These phases are clinically distinguished as an inflammatory or exudative phase for the detachment of deteriorated or necrotic tissues and for wound cleansing, a proliferative phase for the development of granulation tissue, and a differentiation or regeneration phase for maturation, scar formation and epithelization (i.e., cleansing phase, granulation phase, and epithelization phase).

In some embodiments of the present invention, the therapeutic agent is in an inactive form and is rendered active following administration of the composition to target cells or tissues. For example, the agent, upon exposure to light or a change in pH (e.g., due to exposure to a particular intracellular environment) is altered to assume its active form. In these embodiments, the agent may be attached to a protective linker (e.g., photo-cleavable, enzyme-cleavable, pH-cleavable) to make it inactive and become active upon exposure to the appropriate activating agent (e.g., UV light, a cleavage enzyme, or a change in pH).

Thus, the present invention provides a variety of useful therapeutic, diagnostic, and in vitro methods and compositions for delivery of biologically active and therapeutic agents, particularly when associated with solid membrane substrates.

Definitions

To facilitate an understanding of the present invention, a number of terms and phrases are defined below:

As used herein, the term “biocompatible” refers to compositions comprised of natural or synthetic materials, in any suitable combination, that remain substantially biologically unreactive in a host. The term “substantially unreactive” means that any response observed in a host is a subclinical response, i.e., a response that does not rise to a level necessary for therapy.

As used herein, the term “bioerodable” refers to compositions comprised of natural or synthetic materials, in any suitable combination, that are at least partially degraded by biological processes (e.g., enzymatically) or in a biological environment (e.g., within a host or in contact with biological tissues). The rate of degradation of the bioerodable compositions used may vary over time, or be activated by any number of extrinsic or intrinsic factors (e.g., light, heat, radiation, pH, enzymatic or nonenzymatic cleavage, etc.). As used herein, the term “biological tissues” includes cells or tissues in vivo (e.g., cells or tissue of a host) and in vitro (e.g., cultured cells).

As used herein, the term “dendrimer complexes” refers to compositions of dendrimers associated with (e.g., attached covalently or noncovalently) one or more biologically active or therapeutic agents. Dendrimer complexes may incorporate protecting groups or ligands either associated with the dendrimer or associated with the biologically active or therapeutic agents (e.g., lipid moieties, sugar moieties, anionic or nonanionic groups, haptens, etc.). Dendrimer complexes may further comprise or be administered with common pharmaceutical acceptable compositions (e.g., adjutants, excipients, or diluents).

As used herein, the term “surface” when used in the context of either dendrimer complexes or membranes, refers to the surface of these compositions (i.e., the outer regions that are expose to the environment). A composition (e.g., an agent) present at the surface of a denrimer or membrane refers to a composition that is in contact with the dendrimer or membrane, while being at least partially exposed to the environment.

As used herein, the term “biologically active agent” and “therapeutic agent” refers to compositions that possess a biological activity or property having structural (e.g, binding ability), regulatory, or biochemical functions. Moreover, as used herein, the term “agent” refers to biologically active agents and therapeutic agents, except where noted otherwise. Biological activities include activities associated with biological reactions or events in a host that allow the treating, detection, monitoring, or characterization of biological reactions or events. Biological activities include, but are not limited to, therapeutic activities (e.g., the ability to improve biological health or prevent the continued degeneration associated with an undesired biological condition), targeting activities (e.g., the ability to bind or associate with a biological molecule or complex), monitoring activities (e.g., the ability to monitor the progress of a biological event or to monitor changes in a biological composition), imaging activities (e.g., the ability to observe or otherwise detect biological compositions or reactions), and signature identifying activities (e.g., the ability to recognize certain cellular compositions or conditions and produce a detectable response indicative of the presence of the composition or condition). The agents of the present invention are not limited to these particular illustrative examples. Indeed any biologically active agent or therapeutic agent may be used including compositions that deliver or destroy biological materials, cosmetic agents, and the like. The agents may comprise, for example, nucleic acids, antibiotics, chemotherapeutic agents, proteins, and organic or inorganic molecules or compounds. Such agents may or may not further comprise common pharmaceutically acceptable compositions (e.g., adjutants, excipients, or diluents). In preferred embodiments, the agent or agents of the present invention are advantageously administered when associated with dendrimers and acceptable biocompatible or bioerodable membranes. In preferred embodiments of the present invention, the agent or agents are associated with at least one dendrimer (e.g., sequestered or encompassed [i.e., inside] the dendrimer, or covalently or noncovalently attached to the dendrimer surface, etc.).

The terms “pharmaceutically acceptable” or “pharmacologically acceptable,” as used herein, refers to compositions that do not substantially produce, for example, adverse or allergic reactions when administered to host.

The term “agonist,” as used herein, refers to a molecule which, when interacting with a biologically active molecule, causes a change (e.g., enhancement) in the biologically active molecule, which modulates the activity of the biologically active molecule. Agonists include proteins, nucleic acids, carbohydrates, or any other molecules that bind or interact with biologically active molecules. For example, agonists can alter the activity of gene transcription by interacting with RNA polymerase directly or through a transcription factor.

The terms “antagonist” or “inhibitor,” as used herein, refer to a molecule which, when interacting with a biologically active molecule, blocks or modulates the biological activity of the biologically active molecule. Antagonists and inhibitors include proteins, nucleic acids, carbohydrates, or any other molecules that bind or interact with biologically active molecules. Inhibitors and antagonists can effect the biology of entire cells, organs, or organisms (e.g., an inhibitor that slows tumor growth).

The term “change,” as used herein, refers to a change in the biological activity of a biologically active molecule. Modulation can be an increase or a decrease in activity, a change in binding characteristics, or any other change in the biological, functional, or immunological properties of biologically active molecules.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “antisense” is used in reference to DNA or RNA sequences that are complementary to a specific DNA or RNA sequence (e.g., mRNA). Included within this definition are antisense RNA (“asRNA”) molecules involved in gene regulation by bacteria. Antisense RNA may be produced by any method, including synthesis by splicing the gene(s) of interest in a reverse orientation to a viral promoter which permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

The term “transgene” as used herein refers to a foreign gene that is placed into an organism. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots).

As used herein, the term “gene transfer system” refers to any means of delivering a composition comprising a nucleic acid sequence to a cell or tissue. For example, gene transfer systems include, but are not limited to vectors (e.g., retroviral, adenoviral, adeno-associated viral, and other nucleic acid-based delivery systems), microinjection of naked nucleic acid, dendrimers, and polymer-based delivery systems (e.g., liposome-based and metallic particle-based systems). As used herein, the term “viral gene transfer system” refers to gene transfer systems comprising viral elements (e.g., intact viruses and modified viruses) to facilitate delivery of the sample to a desired cell or tissue.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, biolistics, and dendrimers.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “recombinant DNA molecule” as used herein refers to a DNA molecule that is comprised of segments of DNA joined together by means of molecular biological techniques.

As used herein, the terms “contacted” and “incorporated,” when respectively applied to dendrimer complexes and biocompatible or bioerodable membranes, are used to describe the chemical adhesion of the dendrimer complex onto the surface of, or, the physical incorporation of the dendrimer complex into a suitable biocompatible or bioerodable membrane.

As used herein, the terms “contacted” and “exposed,” when applied to target cells or tissues, are used to describe the process by which a composition (e.g., comprising a dendrimer complex and an associated biocompatible or bioerodable membrane) is delivered to a target cell or tissue are placed in contact (e.g., direct contact) with the target cell or tissue.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the term “purified” or “to purify” refers to the removal of contaminants from a sample.

As used herein, the term “medical devices” includes any material or device that is used on, in, or through a patient's body in the course of medical treatment (e.g., for a disease or injury). Medical devices include, but are not limited to, such items as medical implants, wound care devices, drug delivery devices, and body cavity and personal protection devices. The medical implants include, but are not limited to, urinary catheters, intravascular catheters, dialysis shunts, wound drain tubes, skin sutures, vascular grafts, implantable meshes, intraocular devices, heart valves, and the like. Wound care devices include, but are not limited to, general wound dressings, biologic graft materials, tape closures and dressings, and surgical incise drapes. Drug delivery devices include, but are not limited to, drug delivery skin patches, drug delivery mucosal patches and medical sponges. Body cavity and personal protection devices, include, but are not limited to, tampons, sponges, surgical and examination gloves, and toothbrushes. Birth control devices include, but are not limited to, IUD's and IUD strings, diaphragms and condoms.

### DETAILED DESCRIPTION OF THE INVENTION

Certain preferred embodiments of the present invention are described in detail below. The present invention is not limited to these particular described embodiments. The description is provided in the following section: I) Dendrimers Synthesis; II) Biocompatible and Bioerodable Membranes Synthesis; III) Biologically Active and Therapeutic Agents; and IV) Exemplary Embodiments.

I. Dendrimers Synthesis

Dendrimeric polymers have been described extensively (See, Tomalia, Advanced Materials 6:529 [1994]; Angew, Chem. Int. Ed. Engl., 29:138 [1990]; incorporated herein by reference in their entireties). Dendrimer polymers are synthesized as defined spherical structures. Molecular weight and the number of terminal groups increase exponentially as a function of generation (the number of layers) of the polymer. Different types of dendrimers can be synthesized based on the core structure that initiates the polymerization process.

The dendrimer core structures dictate several characteristics of the molecule such as the overall shape, density and surface functionality (Tomalia et al., Chem. Int. Ed. Engl., 29:5305 [1990]). Spherical dendrimers have ammonia as a trivalent initiator core or ethylenediamine (EDA) as a tetravalent initiator core. Recently described rod-shaped dendrimers (Yin et al., J. Am. Chem. Soc., 120:2678 [1998]) use polyethyleneimine linear cores of varying lengths; the longer the core, the longer the rod. Dendritic macromolecules are available commercially in kilogram quantities and are produced under current good manufacturing processes (GMP) for biotechnology applications.

Dendrimers may be characterized by a number of techniques including, but not limited to, electrospray-ionization mass spectroscopy, ¹³C nuclear magnetic resonance spectroscopy, high performance liquid chromatography, size exclusion chromatography with multi-angle laser light scattering, capillary electrophoresis and gel electrophoresis. These tests assure the uniformity of the polymer population and are important for monitoring quality control of dendrimer manufacture for GMP applications and in vivo usage. Extensive studies have been completed with dendrimers and show no evidence of toxicity when administered intravenously in in vivo studies (Roberts et al., J. Biomed. Mat. Res., 30:53 [1996] and Bourne et al., J. Magn. Reson. Imag., 6:305 [1996]).

Numerous U.S. Patents describe methods and compositions for producing dendrimers. Examples of some of these patents are given below in order to provide a description of some dendrimer compositions that may be useful in the present invention, however it should be understood that these are merely illustrative examples and numerous other similar dendrimer compositions could be used in the present invention.

U.S. Pat. No. 4,507,466, U.S. Pat. No. 4,558,120, U.S. Pat. No. 4,568,737, and U.S. Pat. No. 4,587,329 each describe methods of making dense star polymers with terminal densities greater than conventional star polymers. These polymers have greater/more uniform reactivity than conventional star polymers, i.e. 3rd generation dense star polymers. These patents further describe the nature of the amidoamine dendrimers and the 3-dimensional molecular diameter of the dendrimers.

U.S. Pat. No. 4,631,337 describes hydrolytically stable polymers. U.S. Pat. No. 4,694,064 describes rod-shaped dendrimers. U.S. Pat. No. 4,713,975 describes dense star polymers and their use to characterize surfaces of viruses, bacteria and proteins including enzymes. Bridged dense star polymers are described in U.S. Pat. No. 4,737,550. U.S. Pat. No. 4,857,599 and U.S. Pat. No. 4,871,779 describe dense star polymers on immobilized cores useful as ion-exchange resins, chelation resins and methods of making such polymers.

U.S. Pat. No. 5,338,532 is directed to starburst conjugates of dendrimer(s) in association with at least one unit of carried agricultural, pharmaceutical or other material. This patent describes the use of dendrimers to provide means of delivery of high concentrations of carried materials per unit polymer, controlled delivery, targeted delivery and/or multiple species such as e.g., drugs antibiotics, general and specific toxins, metal ions, radionuclides, signal generators, antibodies, interleukins, hormones, interferons, viruses, viral fragments, pesticides, and antimicrobials.

Other useful dendrimer type compositions are described in U.S. Pat. No. 5,387,617, U.S. Pat. No. 5,393,797, and U.S. Pat. No. 5,393,795 in which dense star polymers are modified by capping with a hydrophobic group capable of providing a hydrophobic outer shell. U.S. Pat. No. 5,527,524 discloses the use of amino terminated dendrimers in antibody conjugates.

The use of dendrimers as metal ion carriers is described in U.S. Pat. No. 5,560,929. U.S. Pat. No. 5,773,527 discloses non-crosslinked polybranched polymers having a comb-burst configuration and methods of making the same. U.S. Pat. No. 5,631,329 describes a process to produce polybranched polymer of high molecular weight by forming a first set of branched polymers protected from branching; grafting to a core; deprotecting first set branched polymer, then forming a second set of branched polymers protected from branching and grafting to the core having the first set of branched polymers, etc.

U.S. Pat. No. 5,902,863 describes dendrimer networks containing lipophilic organosilicone and hydrophilic polyanicloamine nanscopic domains. The networks are prepared from copolydendrimer precursors having PAMAM (hydrophilic) or polyproyleneimine interiors and organosilicon outer layers. These dendrimers have a controllable size, shape and spatial distribution. They are hydrophobic dendrimers with an organosilicon outer layer that can be used for specialty membrane, protective coating, composites containing organic organometallic or inorganic additives, skin patch delivery, absorbants, chromatography personal care products and agricultural products.

U.S. Pat. No. 5,795,582 describes the use of dendrimers as adjutants for influenza antigen. Use of the dendrimers produces antibody titer levels with reduced antigen dose. U.S. Pat. No. 5,898,005 and U.S. Pat. No. 5,861,319 describe specific immunobinding assays for determining concentration of an analyte. U.S. Pat. No. 5,661,025 provides details of a self-assembling polynucleotide delivery system comprising dendrimer polycation to aid in delivery of nucleotides to target site. This patent provides methods of introducing a polynucleotide into a eukaryotic cell in vitro comprising contacting the cell with a composition comprising a polynucleotide and a dendrimer polycation non-covalently coupled to the polynucleotide.

Dendrimer-antibody conjugates for use in in vitro diagnostic applications has previously been demonstrated (Singh et al., Clin. Chem., 40:1845 [1994]), for the production of dendrimer-chelant-antibody constructs, and for the development of boronated dendrimer-antibody conjugates (for neutron capture therapy); each of these latter compounds may be used as a cancer therapeutic (Wu et al., Bioorg. Med. Chem. Lett., 4:449 [1994]; Wiener et al., Magn. Reson. Med. 31:1 [1994]; Barth et al., Bioconjugate Chem. 5:58 [1994]).

Some of these conjugates have also been employed in the magnetic resonance imaging of tumors (Wu et al., [1994] and Wiener et al., [1994], supra). Results from this work have documented that, when administered in vivo, antibodies can direct dendrimer-associated therapeutic agents to antigen-bearing tumors. Dendrimers also have been shown to specifically enter cells and carry either chemotherapeutic agents or genetic therapeutics. In particular, studies show that cisplatin encapsulated in dendrimer polymers has increased efficacy and is less toxic than cisplatin delivered by other means (Duncan and Malik, Control Rel. Bioact. Mater. 23:105 [1996]).

Dendrimers have also been conjugated to fluorochromes or molecular beacons and shown to enter cells. They can then be detected within the cell in a manner compatible with sensing apparatus for evaluation of physiologic changes within cells (Baker et al., Anal. Chem. 69:990 [1997]). Finally, dendrimers have been constructed as differentiated block copolymers where the outer portions of the molecule may be digested with either enzyme or light-induced catalysis (Urdea and Hom, Science 261:534 [1993]). This would allow the controlled degradation of the polymer to release therapeutics at the disease site and could provide a mechanism for an external trigger to release the therapeutic agents.

Preferred dendrimer complexes of the present invention are constructed and associated with biocompatible or bioerodable membranes that allow the option of storing and using the dendrimer complexes in arid environments while retaining the ability to deliver biologically active or therapeutic agents.

In some embodiments of the present invention, the preparation of PAMAM dendrimers is performed according to a typical divergent (building up the macromolecule from an initiator core) synthesis. It involves a two-step growth sequence that consists of a Michael addition of amino groups to the double bond of methyl acrylate (MA) followed by the amidation of the resulting terminal carbomethoxy, —(CO₂CH₃) group, with ethylenediamine (EDA).

In the first step of this process, ammonia is allowed to react under an inert nitrogen atmosphere with MA (molar ratio: 1:4.25) at 47° C. for 48 hours. The resulting compound is referred to as generation=0, the star-branched PAMAM tri-ester. The next step involves reacting the tri-ester with an excess of EDA to produce the star-branched PAMAM tri-amine (G=0). This reaction is performed under an inert atmosphere (nitrogen) in methanol and requires 48 hours at 0° C. for completion. Reiteration of this Michael addition and amidation sequence produces generation=1.

Preparation of this tri-amine completes the first full cycle of the divergent synthesis of PAMAM dendrimers. Repetition of this reaction sequence results in the synthesis of larger generation (G=1-5) dendrimers (i.e., ester- and amine-terminated molecules, respectively). For example, the second iteration of this sequence produces generation 1, with a hexa-ester and hexa-amine surface, respectively. The same reactions are performed in the same way as for all subsequent generations from 1 to 9, building up layers of branch cells giving a core-shell architecture with precise molecular weights and numbers of terminal groups as shown above. Carboxylate-surfaced dendrimers can be produced by hydrolysis of ester-terminated PAMAM dendrimers, or reaction of succinic anhydride with amine-surfaced dendrimers (e.g., full generation PAMAM, POPAM or POPAM-PAMAM hybrid dendrimers). Various dendrimers can be synthesized based on the core structure that initiates the polymerization process. These core structures dictate several important characteristics of the dendrimer molecule such as the overall shape, density, and surface functionality (Tomalia et al., Angew. Chem. Int. Ed. Engl., 29:5305 [1990]). Spherical dendrimers derived from ammonia possess trivalent initiator cores, whereas EDA is a tetra-valent initiator core. Recently, rod-shaped dendrimers have been reported which are based upon linear poly(ethyleneimine) cores of varying lengths the longer the core, the longer the rod (Yin et al., J. Am. Chem. Soc., 120:2678 [1998]).

The dendrimers may be characterized for size and uniformity by any suitable analytical techniques. These include, but are not limited to, atomic force microscopy (AFM), electrospray-ionization mass spectroscopy, MALDI-TOF mass spectroscopy, ¹³C nuclear magnetic resonance spectroscopy, high performance liquid chromatography (HPLC) size exclusion chromatography (SEC) (equipped with multi-angle laser light scattering, dual UV and refractive index detectors), capillary electrophoresis and get electrophoresis. These analytical methods assure the uniformity of the dendrimer population and are important in the quality control of dendrimer production for eventual use in in vivo applications.

In preferred embodiments of the present invention, the dendrimer complexes comprise generation (G) 5, 7, and 9 of EDA core dendrimers with molar masses of 28,826, 116,493, and 467,162 Da, and numbers of surface charges (amine groups) of 128, 512, 2,048 (respectively).

In particularly preferred embodiments, the dendrimer-based delivery systems of the present invention are associated with biocompatible or bioerodable membranes and materials. In some of these embodiments, one or more dendrimer are associated with the biocompatible or bioerodable membranes and materials. In other embodiments, one or more biologically active or therapeutic agents are associated with one or more dendrimers. Additionally, in some embodiments, one or more pharmacologically accepted agents are associated with one or more dendrimers or one or more biologically active or therapeutic agents.

In some embodiments, the dendrimer complexes or membrane compositions of the present invention further comprise agent(s) that promote disassociation or distribution of dendrimers complexes from the associated membranes, thus, enhancing delivery or expression of biologically active or therapeutic agents to target cells or tissues. In still other embodiments, other additional agents are provided with the dendrimers or membranes to facilitate delivery, either locally, or more globally. For example, it was discovered during development of the present invention that β-cyclodextrins (β-CD) when associated with the dendrimer complexes of the present invention promotes the even distribution of the complexes and enhances transfection effectiveness. However, the present invention is not limited to any particular means that promotes or enhances the dendrimer-based delivery or expression of biologically active or therapeutic agents, indeed, in some embodiments, the present invention contemplates external means that aid in release of dendrimers and/or agents associated with the dendrimers (e.g., heat, light, ultrasonic energy, and the like).

In preferred embodiments of the present invention, libraries of individual dendrimers comprising the above functionalities are created for use in generating any desired dendrimer-based complexes. For example, libraries of dendrimers each containing one of a host of therapeutic agents are created. The same procedure is conducted for target agents, and the like. Such libraries provide the ability to mix-and-match components to generate the optimum therapy or diagnostics or diagnostic complexes for a desired application. The dendrimer-based complexes may be generated rationally, or may be generated randomly and screened for desired activities. Thus, the present invention provides non-toxic systems with a wide range of therapeutic and diagnostic uses.

II. Biocompatible and Bioerodable Membranes Synthesis

In preferred embodiments of the present invention, the dendrimer complexes are associated with biocompatible or bioerodable membranes. Biocompatible or bioerodable membranes have been described extensively. For example, biocompatible or bioerodable membrane materials composed of polymers such as poly(DL-lactide-co-glycolide) (PLGA), poly(beta-hydroxylkanoates) (PHA), poly(L-lysine citramide imide) (PLCAI), polyethylenterephtalate fabrics (PET), derivatives of cellulose, collagen, fibronectin, calcium sulfates, carbon, chitin (chitosan), and others, have been tested for their suitability to serve as platforms for delivering various pharmaceuticals, as tissue scaffolds, and generally as biocompatible and bioerodable materials (See e.g., 19; 23; Pavlova et al., Biomaterials 14(13):1024 [1993]; Sottosanti, Compendium 13(3):226-8, 230, 232-4 [1992]; Brandl et al., Adv Biochem Eng Biotechnol; 41:77 [1990]; Braunegg et al., J Biotechnol 65(2-3):127 [1998]; Gac et al., J Drug Target 7(5):393 [2000]; Mayer et al., J Controlled Release 64(1-3):81 [2000]; Madihally and Matthew, Biomaterials 20(12):1133 [1999]; herein incorporated by reference in their entireties). A number of materials tailored to wound covering are also known (See e.g., U.S. Pat. Nos. 4,965,128; 4,161,948; and EP0099758; herein incorporated by reference in their entireties).

Biocompatible or bioerodable membrane materials suitable for association to the dendrimer complexes of the present invention may be selected from any one or more of the above mentioned compositions, or from other suitable compositions. Indeed, any membrane capable of being associated with the dendrimers of the present invention, while allowing the desired use (e.g., delivery of an agent to a tissue), is contemplated.

In some embodiments of the present invention, one or more dendrimer complexes are associated with a suitable biocompatable or bioerodable membrane. In other embodiments, one or more dendrimer complex provides one or more similar or dissimilar biologically active or therapeutic agents. In other embodiments, dendrimers with one or more layers are provided for associating biologically active or therapeutic agents.

The present invention also provides dendrimer complexes suitable for delivering biologically active or therapeutic agents at biologically important (e.g., cued to biological processes), or other desired times. In some of these embodiments, an endogenous or exogenous cue may be provided in association with the compositions of the present invention that promotes or retards the delivery of biologically active or therapeutic agents (e.g., UV light, heat, radiation, ultrasonic energies, enzymes, inorganic chemicals and compounds, and the like). For example, agents may be attached to dendrimers with linker groups that are cleaved upon exposure to any of the above endogenous or exogenous cues. In such embodiments, the present invention provides systems for time release delivery of agents.

In still further embodiments, any one or more of the components of compositions of the present invention (e.g., a dendrimer, an agent associated with the dendrimer, and a membrane associated with dendrimer and agent) may further comprise a pharmacologically accepted agent (e.g., adjuvants, excipients, diluents, and the like).

In certain of those embodiments directed to wound healing, the dendrimer complexes are associated with one or more occlusive wound dressings. In other embodiments directed to wound healing, the compositions and methods of the present invention additionally comprise pharmacological agents that promote wound healing.

III. Biologically Active and Therapeutic Agents

A wide range of biologically active and therapeutic agents find use with the present invention. Any agent that can be associated with a dendrimer may be delivered using the methods, systems, and compositions of the present invention. In preferred embodiments of the present invention, the dendrimer-based delivery systems are utilized for promoting wound healing by delivering nucleic acids, or proteins associated with wound healing or that promote or prevent vascularization of tissues (e.g., grafted tissues and transplanted organs). In some embodiments, the dendrimer-based delivery systems of the present invention are used to delivery biologically active or therapeutic agents to kertinocytes and related tissues, and to cervical cells and tissues. The wound healing methods and compositions of the present invention in some embodiments may also be directed to wounds and lesions of the integument, while other embodiments are directed to healing internal wounds and lesions. Thus, the present invention provides compositions and methods for the dendrimer-based delivery of biologically active and therapeutic agents to target cells and tissues both in vivo and in vitro.

In some embodiments, the biologically active or therapeutic agents of the present dendrimer-based delivery systems comprise nucleic acid sequences. In certain embodiments directed to wound healing the dendrimer-based delivery systems comprise nucleic acid sequences encoding cellular mediators and growth factors, including angiogneic factors (e.g., cytokines [e.g., interleukins], tumor necrosis factor alpha [TNF-α], basic fibroblast growth factor [bFGF], epidermal growth factor [EGF], platelet derived growth factor [PDGF], and transforming growth factors alpha and beta [TGF-α, and TGF-β], etc). In some other embodiments, the dendrimer complexes of the present invention are associated with one or more antiangiogenic agents (e.g., suramin, retanoids, interferons, antiestrogens, kringle 5 peptide/fragment, etc.). Numerous references discuss angiogenesis/antiangiogenesis (See e.g., Folkman et al., Science, 235:442 [1987]; Folkman et al., Journ. of Biol. Chem., 267(16):10931 [1992]; Fidler et al., Cell, 79:185 [1994]; Folkman, New Eng. J. Med., 333(26):1757 [1995]). In certain embodiments, the dendrimer complexes of the present invention are associated with one or more anti-inflammatory agents or factors (e.g., non-steroidal [e.g., indomethacin, naproxen, ibuprofen, ramifenazone, piroxicam, and the like] and steroidal [e.g., cortisone, dexamethasone, fluazacort, hydrocortisone, prednisolone, prednisone, and the like]).

In other embodiments, one or more of the aforementioned proteins, or other purified proteins, may be associated with the dendrimer-based deliver systems of the present invention. In certain other embodiments directed to wound healing, the dendrimer-based delivery systems comprise drugs and/or pharmacological agents that promote wound healing and/or promote or prevent vascularization of tissue.

In yet other embodiments directed to wound healing, one or more biologically active or therapeutic agents that promote nerve growth are associated with the dendrimer complexes (e.g., nerve growth factors [NGFs]). NGFs are neurotropic proteins that play a critical role in the development and maintenance of sympathetic and embryonic sensory neurons (See e.g, Levi-Montalcini, In Vitro Cell. Devel. Biol. 23:227 [1987]).

In some embodiments, the biological active and therapeutic agents may be associated with the dendrimers of the present invention in any biologically effective combination or amount. Thus, in some embodiments, biologically active or therapeutical agents with known beneficial synergies may be associated with one or more dendrimers. In some embodiments, vaccinating agents (e.g., compositions that promote or enhance an immunological response in a host) are associated with the dendrimers of the present invention.

In order to assess the suitability of any particular biologically active or therapeutic agent (e.g., agents that regulate wound healing, vascularization, epithelization, transfection, and expression of transgenes, etc.) contemplated for association with the dendrimer complexes of the present invention and use for a particular application, a simple, efficiency assay is provided. In brief, the assay comprises 1) providing one or more compositions to be tested for their suitability as a biologically active or therapeutic agents when associated with one or more dendrimer complexes of the present invention; 2) associating the composition to be tested with one or more dendrimers (or associating the compound to be tested with a suitable biocompatable or bioerodable membrane); 3) associating the dendrimer complex with any attached compound to be tested to a suitable membrane (e.g., biocompatable or bioerodable membrane); 4) contacting the dendrimer complex and associated biocompatable or bioerodable membrane to a cell or tissue (e.g., in vivo testing in an animal, in vitro testing, etc.); and 5) detecting a change in the cell or tissue or a change in a host comprising the cell or tissue (e.g., a phenotypic change, etc.). If the change in the cell, tissue, or host is a desired change, the composition is deemed suitable. For example, for therapeutic applications, if a candidate composition provides a detectable improvement in at least one symptom of a disease or condition (e.g., a suitable wound healing candidate composition increases the rate of wound healing), the composition is deemed suitable for such therapeutic applications. The assay provided is thus useful for determining the suitability of associating one or more potential biologically active or therapeutic agents with one or more dendrimers and membranes of the present invention for use in any desired application.

For example, the present invention demonstrates that PAMAM dendrimer complexes when associated with biologically active or therapeutic agents (e.g., nucleic acids) and further associated with suitable biocompatable or bioerodable (e.g., PLGA or collagen) membranes, effectively delivered (e.g., transfected) target cells and tissues in vivo and in vitro.

IV. Exemplary Embodiments

The direct topical delivery of nucleic acids and biologically active agents and into skin cells holds great promise as a human gene therapy technique, as a reservoir for transgene products, and for diagnostic applications. However, problems associated with topical gene delivery, and gene therapy in general (See e.g., Ye et al., Mol Med Today 4:431 [1998]; Jane et al., Annals of Med 30:413 [1998]), are further complicated by the protective structure and function of the skin (e.g., skin is not easily penetrated by charged macromolecules including nucleic acids). Liposome-DNA formulations for the perifollicular delivery and expression of therapeutic DNA sequences have met with limited success. (See e.g., Niemiec et al., J. Pharm. Sci. 86:701 [1997]). For example, liposomal-based nucleic acid delivery compositions are not effective when dried prior to their administration. In contrast, the present invention provides dendrimer-based biologically active and therapeutic agent delivery complexes that efficiently transfect cultured cells and that find use as carriers for in vitro and in vivo applications when associated with biocompatible or bioerodable membranes.

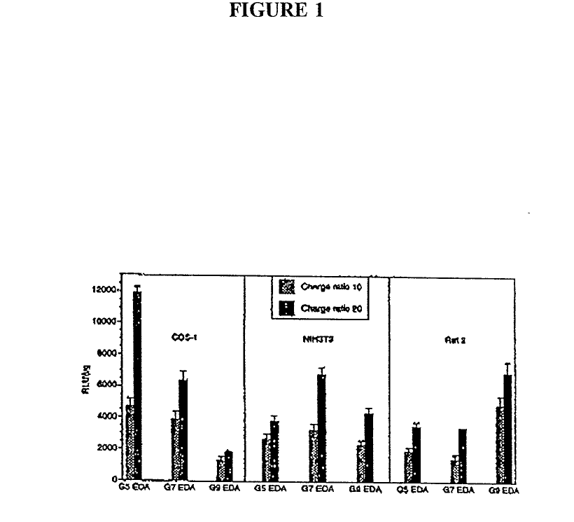

In some embodiments, poly(DL-lactide-co-glycolide) (PLGA) polymer membranes (See e.g., Example 2) were used with the dendrimer complexes of the present invention and demonstrated the ability to transfect cells in vitro. pCF1-Luc (encoding firefly luciferase) and pEGFP1 (encoding green fluorescent protein), plasmid DNA was used to detect and assess efficiency and frequency of transfection (See e.g., Example 3). Dendrimer/nucleic acid complexes were generated at 0.1 mg/ml DNA with E5 EDA, E7 EDA and E9 EDA at the charge ratio 10 and 20. Small, (approximately 10 mm²) fragments of the PLGA film were incubated with the water based suspension of DNA/dendrimer complexes and then air-dried in sterile conditions (See e.g., Example 4). Cultures of adherent NIH 3T3, COS-1 and Rat2 cells (See e.g., Example 5) were incubated with PLGA membranes coated with DNA/dendrimer complexes. All cell lines expressed luciferase, indicating successful transfection as shown in FIG. 1. FIG. 1 shows luciferase expression in COS-1, NIH 3T3, and Rat 2 cells transfected with dendrimer/DNA complexes coated on the surface of PLGA membranes. Luciferase expression is presented as relavitve light units/μg of total protein. Columns represent mean values of three repeats (+/−SD). No cyctoxic effects were observed. Cells successfully transfected with pEGFP1, identified using fluorescent microscopy, were found on the entire surface of the culture plates with a frequency of 1-5%. This result indicates that dendrimer complexes can dissociate from PLGA membranes and retain transfectional activity.

In some embodiments, the dendrimer complexes of the present invention are associated both into and onto the surface of collagen membranes (See e.g., Example 2). In preferred embodiments, collagen membranes are associated with the dendrimer complexes for topical delivery of agents to keratinocytes. Furthermore, in other embodiments, it is contemplated that addition of fibronectin-like peptides to the collagen membranes enhances the adherence of the dendrimer complexes to target cells and tissues. Thus, dendrimer/nucleic acid complexes generated in various DNA concentrations and charge ratios were coated on the surface collagen/fibronectin-peptide membranes and then tested for their ability to delivery nucleic acids to target cells. Analysis of the release of the radioactive DNA indicated the immediate dissociation of the dendrimer complexes from the collagen membranes.

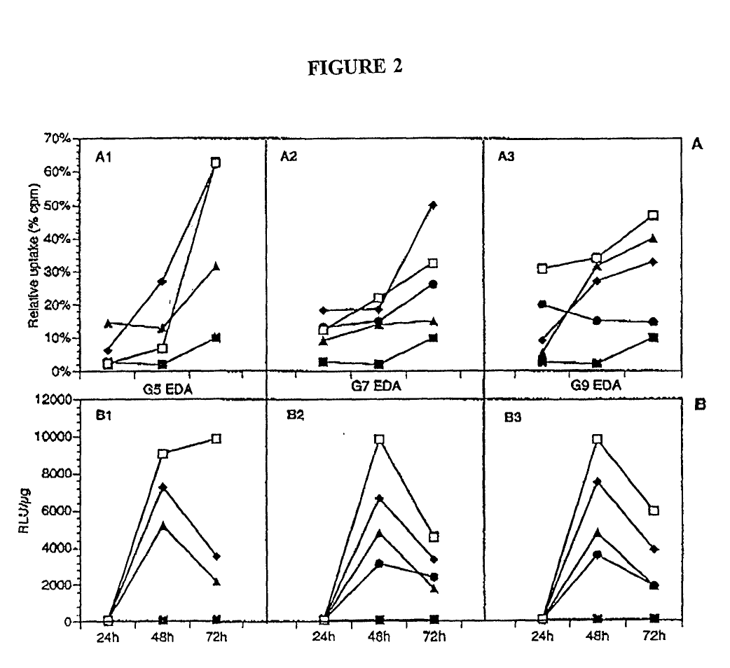

The ability to deliver biologically active and therapeutic agents via dendrimer complexes incorporated directly into collagen/fibronectin membranes was determined. The presence of the dendrimers complexes retarded the release of the radioactive labeled DNA, the level of which did not significantly increase during 72 hr of incubation. However, intracellular DNA uptake by normal human foreskin fibroblast (NHF1 cells) cultured on the surface of the membranes was enhanced and prolonged in the presence of dendrimer complexes and seemed to be more efficient at dendrimer/nucleic acid charge ratios of between 10 and 20, compared to charge ratios of 0. 1 or 1 (as shown in FIG. 2A). Moreover, the efficiency of transfection increased as a function of the excess of cationic dendrimer (charge ratios >5) present in the membrane. The expression of the reporter luciferase gene in most cells peaked at 48 hr after initiating the cell cultures on the surface of the membranes (FIG. 2B), while increasing amounts of cell associated radioactivity were observed at 72 hr, indicating continuous uptake of dendrimer complexed nucleic acid (FIG. 2A). Cells cultured on the membranes with naked plasmid DNA did not significantly express the luciferase transgene. In FIG. 2, DNA uptake (A) and transfection (B) of NBF1 cells cultured on the collagen/fibronectin membranes containing incorporated dendrimer/DNA complexes. Panels A1 and B1 show uptake and expression, respectively, using complexes formed using G5 dendrimers. Panels A2 and B2 show uptake and expression, respectively, using G7 EDA dendrimers. Panels A3 and B3 show uptake and expression, respectively, using complexes formed using G9 EDA dendrimers. Values represent the mean of three repeats, SD does not exceed 15% of total (closed boxes)—naked DNA; (closed circles)—dendrimer/DNA at charge ratio 0.1; (closed triangles)—dendrimer/DNA at charge ratio 1; (closed diamonds)/DNA at charge ratio 10; (open boxes)—dendrimer/DNA at charge ration 20.

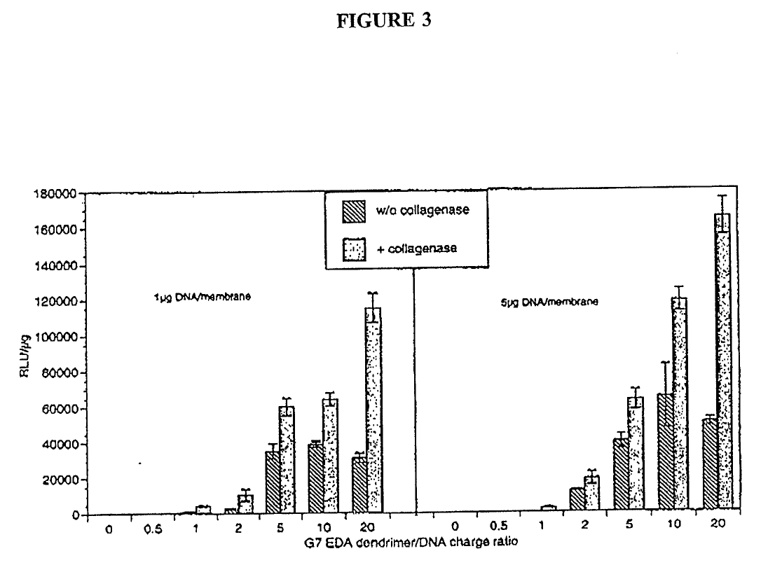

The effects of treating collagen membranes with collagenase to affect the increase the efficiency of transfection by associated dendrimer/nucleic acid complexes (See e.g., Examples 4 and 7) incorporated into the membranes was determined. While an understanding of the mechanisms is not necessary for practicing the present invention and the present invention is not limited to any particular mechanism, it is believed that exposing the collagen membranes to collagenase increases the accessibility of the dendrimer complexes to the cells, thus, increases transfection efficiency. Normal human foreskin fibroblasts (NBF1 cells) were seeded on the membranes containing broad range (0.5 to 20) dendrimer/DNA charge ratio of G7 EDA/pCF1-Luc DNA complexes (See e.g., Examples 5 and 6). When the collagen membranes were preincubated with collagenase, expression of luciferase transgene gene increased 2 to 3 fold depending on the charge ratio of the dendrimer/nucleic acid complexes (FIG. 3). In FIG. 3, the effect of the preincubation with collagenase on the efficiency of COS cells transfection using G7 EDA dendrimer/DNA complexes incorporated into collage membranes. Columns represent the mean of three repeats (+/−SD). Cell viability ranged from 80 to 100% of control. These results indicate that collagen proteolysis of the collagen membranes leads to the greater accessibility of dendrimer complexes to the target cells and increases the efficiency of the in situ transfections by the methods and compositions of the present invention.

Thus, in some embodiments, dendrimer complexes, when coated on the surface of moderately charged PLGA or collagen/fibronectin membranes, even when desiccated, successfully delivery of transgenes to cultured cells and also successfully dissociate from the membranes and transfect surrounding cells. The efficiency of transfections is a function of both the nucleic acid concentration and the dendrimer/DNA charge ratio. In some embodiments, the methods and compositions of the present invention further comprise collagenase to aid the disassociation of dendrimer complexes from the collagen/fibronectin membranes. In some embodiments, the dendrimer complexes also retained a substantial degree of activity when incorporated directly into a matrix of polymerized collagen. This suggests that prolonged release kinetics and/or controllable rates of release can be achieved when similar membranes are applied to skin and other organ systems for in vivo transfection.

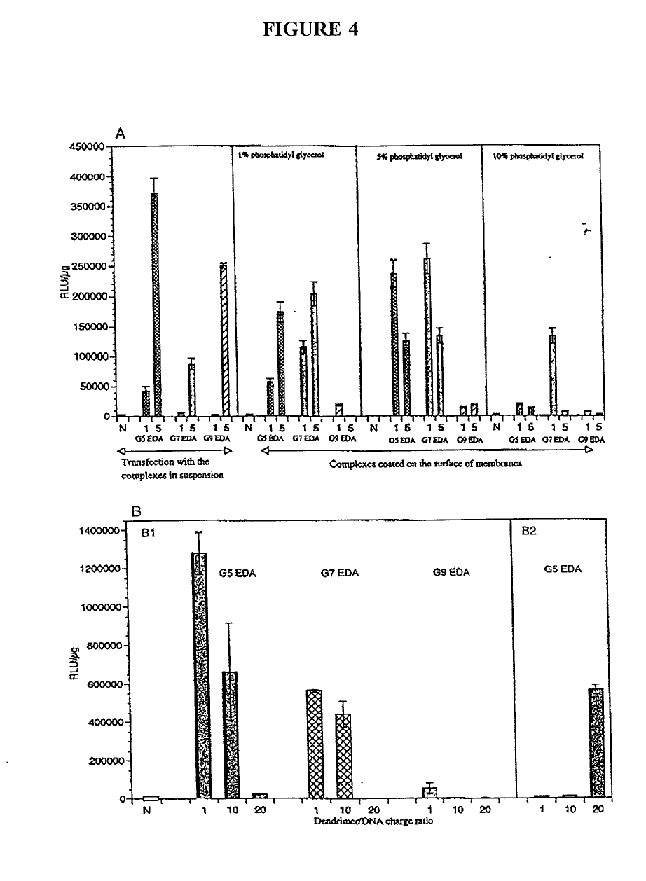

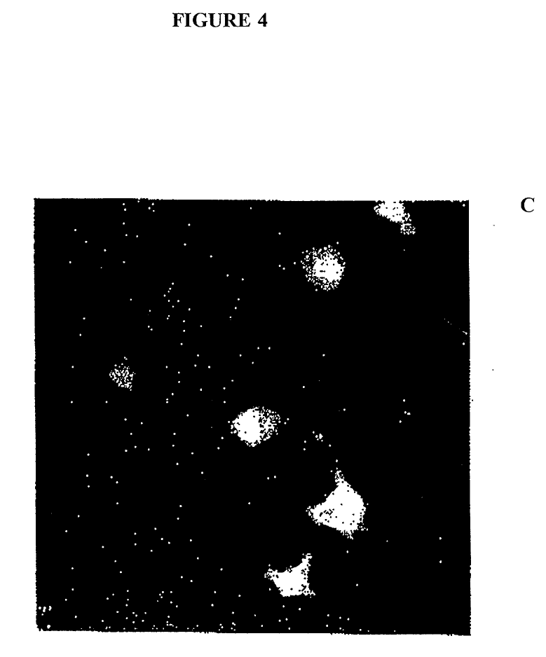

In another series of experiments conducted during the development of the present invention, the effects of phosphatidylglycerol on the in situ transfection were observed. Different concentrations of the anionic lipid phosphatidylglycerol (PG) were incorporated into collagen/fibronectin membranes in order to increase the anionic surface charge differential between membranes and DNA/dendrimer complexes. In contrast to unmodified collagen membranes, DNA/complexes were gradually released from the surface of the PG containing membranes over a period of 48 hr. pCF1-Luc DNA complexed with G5, G7 and G9 EDA dendrimer at dendrimer/DNA charge ratio 1 and 5 was coated onto the surface of collagen membranes containing 1, 5, and 10% phosphatidyl glycerol. In situ transfection of COS-1 cells resulted the expression of luciferase. Presence of 1 and particularly 5% PG resulted in an efficiency of transfection of G5 and G7 dendrimer/DNA complexes comparable (50 to 75% depending on the charge ratio) to the standard solution-based transfection controls. A further increase of the PG concentration up to 10% inhibited transfection, in particular, at the charge ratio 5 (FIG. 4A). FIG. 4A shows transfection efficiencies in COS-1 were compared between complexes used in solution versus complexes coated onto the surface of collagen membranes containing 1, 5, or 10% (wt %) of PG. Columns represent the mean of three repeats (+/−SD). N indicates no dendrimers, 1 and 5 indicate dendrimer/DNA charge ratio.

Extending the range of dendrimer/DNA complexes up to 20 did not further enhance transfection (FIG. 4B). FIG. 4B shows NHF-1 cells were transfected with complexes formed using G5, G7, and G9 EDA dendrimers at dendrimer/DNA charge ratios of 1, 10, or 20. Columns represent the mean of three repeats. B1 shows results of experiments using dendrimer/DNA complexes coated on collagen/fibronectin/5% phosphatidyl glycerol (PG) membranes. B2 shows the results obtained using dendrimer/DNA complexes coated on collagen/fibronectin membranes without PG. As previously observed, the PG containing membrane supported transfection was more effective in lower dendrimer/DNA charge ratios (e.g., 1), which are not efficient for the transfections in solution or on the surface collagen/fibronectin membranes without PG. Naked pCF1DNA (Luc) DNA coated on the surface of PG containing membranes used as a control, did not efficiently transfect COS-1 and NHF1 cells.

Thus, in some embodiments, the modification of the membranes by an anionic component (PG) results in the increase of the charge differential between dendrimer/DNA complexes and the membranes. These results indicate that membrane supported dendrimer based DNA delivery can achieve transfection levels comparable to solution based delivery methods, but at much lower dendrimer/DNA charge ratios.

Primary human keratinocytes (PHEK) have proven to be a good in vitro model for skin transfection since it is hard to consistently obtain quantifiable levels of reporter gene expression in these cells. However, using methods and compositions of the present invention, in situ transfection of keratinocytes cultured on collagen-PG membranes with pEGFP1 plasmid complexed with E5 EDA dendrimers resulted in the expression of green fluorescent protein in 15-20% of cells present on the surface of the membranes. To obtain this frequency of transgene expression, cells were incubated with 10 pg/ml of recombinant human EGF (FIG. 4C). FIG. 4C shows fluorescent photomicrograph showing GFP expression in PHEK transfected with dendrimer/pCF1 GFP complexes using G5 EDA dendrimer and a charge ratio of 1. No transfected cells were observed on the membranes coated with naked DNA (Magnification 200×).

The ability of dendrimer complexes, when associated with collagen-PG membranes, to deliver biologically active and therapeutic agents in vivo on test animals was also determined. For in vivo transfection of hairless mice, collagen-PG membranes (PG 5 wt %) were coated with 50 or 100 μg of pCF1CAT DNA alone or complexed with G5 EDA dendrimers at 0.1, 1, or 10 dendrimer/DNA charge ratios. After drying, the membranes were used for in vivo delivery of the complexed DNA to the denuded skin of hairless mice (See e.g., Example 6).

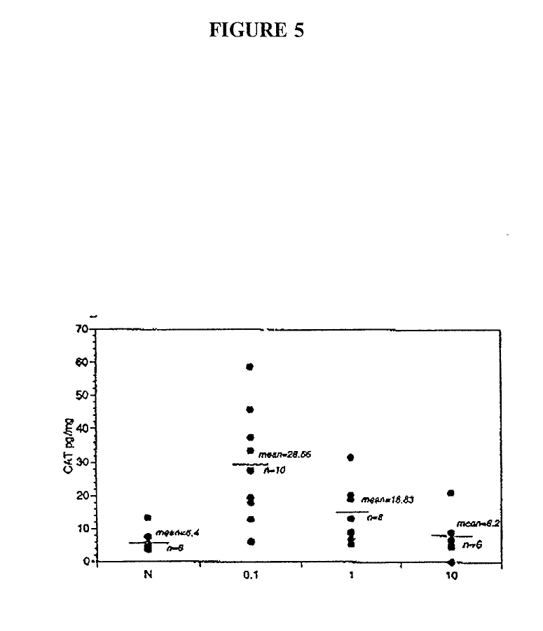

At the time of harvest, the collagen membranes had been completely reabsorbed by the host skin and no residual membrane could be detected. Skin biopsies were collected after 24 hours, homogenized and level of expression of chloramphenicol transacetylase (CAT) was determined using CAT-ELISA (See e.g., Example 9-12). Membrane mediated transfections with uncomplexed plasmid did not result in significant expression of the transgene. CAT expression reaching 50-250 pg/mg of skin biopsy homogenates was obtained with complexes formed at 0.1 G5/DNA charge ratios and 50 μg DNA (DNA concentration during complex formation: 0.05 mg/ml). While an understanding of the mechanism is not necessary to practive the present invention and the present invention is not limited to any particular mechanism, this difference in the transfection efficiency/expression may be the result of dendrimer/DNA precipitates that are generated when complexes are formed in the presence of DNA in concentrations >0.01 mg/ml as well as presence of epidermal cells capable of expressing transfected DNA. CAT expression in the mouse skin transfected with collagen-PG membrane supported dendrimer/DNA indicate that complexes are most effective at charge ratios of (<1) and increase transgene expression 6-8 fold above uncomplexed DNA (FIG. 5). Precipitation and aggregation of dendrimer/DNA complexes does not occur at lower DNA concentrations. The time course of the expression revealed the transient nature of the expression with peak at 24 hours. Levels of the transgene expression declined approximately 70% by 48 hr post-application. FIG. 5 shows CAT expression in hairless mouse skin following topical delivery of dendrimer/pCF1CAT complexes using PG/collagen/fibronectin membranes. The mean value of CAT expression from all skin biopsies obtained in an individual animal was plotted (dots represent individual animals). Mean values from each treatment group (charge ratios; N, 0.1, 1, and 10) are indicated by horizontal lines.

Thus, the dendrimer complexes of the present invention when administered to the denuded skin of hairless mice resulted in appreciable expression of the transgenic. In contrast, naked DNA on the surface of the membranes did not efficiently enter and was not expressed in the dermal cells. Moreover, the present invention indicates that transfection is a property specific to the assembled membranes and not the individual components per se. In support of this conclusion is that attempts to obtain in vitro transfection using Lipofectamine- or Lipofectin-DNA formulations coated and dried on the surface of PLGA and collagen membranes were unsuccessful. Accordingly, the present invention provides dendrimer complexes associated with biocompatible or bioerodable membranes that can be desiccated while retaining transfectivity. Current liposomal transfection systems do not have this same stability to withstand desiccation or lyophilization.

Thus, the methods and compositions of the present invention find use as therapeutics (e.g., promoting healing in both acute and chronic wounds as well as disease and lesions). The methods and compositions of the present invention also find use as diagnostic applications (e.g., introducing an agent and tracking the agents distribution/localization in a cell or tissue). In diagnostics applications the present invention may further comprise one or more tracking agents (e.g., radioisotopes, clorometric agents, antigenic determinants, marker genes, and the like). Other embodiments of the present invention provide drug screens. For example, arrays of dendrimer complexes comprising a plurality of different agents are contacted with a target tissue or cells (e.g., cells in a multichamber plate) and local responses are detected. In other embodiments, agents are delivered using the membranes of the present invention in conjunction with candidate drug compounds to determine the effect of the compound in the presence or absence of the delivered agent.

In preferred embodiments, the methods and compositions of the present invention provide effective in vitro transfection reagents. In still other preferred embodiments, the methods and compositions of the present invention provide effective ex vivo transfection reagents (e.g., transfection of tissue grafts and transplants, or transfection of cell line and tissues for non-clinical uses). In certain particular embodiments, skin grown in vivo or obtained ex vivo is transfected and then grafted to a host (e.g., a burn patient).

In yet other embodiments, the methods and compositions of the present invention provide effective delivery systems for association with medical devices. In certain of these embodiments, the present invention provides delivery of anti-inflammatory agents, anti-pathogen agents, etc.

The present invention is not limited by the route of administration. Contemplated routes of administration include, but are not limited to, endoscopic, intratracheal, intralesion, percutaneous, intravenous, subcutaneous, and intratumoral administration. Experiments conducted during the development of the present invention have used both surface coating or incorporation of dendrimer complexes into poly(DL-lactide-co-glycolide) (PGLA) or collagen-based biocompatible membranes as systems to facilitate transfection of dermal cells in vitro and in vivo using skin as a target organ.

### EXAMPLES

The following examples serve to illustrate certain preferred embodiments and aspects of the present invention and are not to be construed as limiting the scope thereof. In the experimental disclosure which follows, the following abbreviations apply: eq (equivalents); μ(micron); M (Molar); μM (micromolar); mM (millimolar); N (Normal); mol (moles); mmol (millimoles); μmol (micromoles); nmol (nanomoles); g (grams); mg (milligrams); μg (micrograms); ng (nanograms); L (liters); ml (milliliters); μl (microliters); cm (centimeters); mm (millimeters); μm (micrometers); nM (nanomolar); ° C. (degrees Centigrade); PBS (phosphate buffered saline); and RT (room temperature).

### Example 1

### Dendrimer Synthesis

Dendrimers were synthesized as described by Tomalia et al. (See e.g., Tomalia et al, Agnew Chem. Int. Ed. Engl. 29:138 [1990]; See also, Frechet, Science 263:1710 [1994]). Studies were performed with generation (G) 5, 7, and 9, EDA core PAMAM dendrimers with molar masses of 28,826, 116,493, and 467,162 Da and numbers of primary surface amine groups surface charges (amine groups) of 128, 512, 2,048 respectively.

### Example 2

Preparation of Biocompatible Membranes

This example describes methods used to prepare some of the membranes of the present invention. Poly(DL-lactide-co-glycolide) (PLGA) membranes were prepared by dissolving poly(DL-lactide-co-glycolide) (75:25 M.W. 75,000-120,000, Sigma) monomer in chloroform (10% wt/vol) and pouring the solution onto the surface of sterile siliconized Pyrex dishes. Chloroform was evaporated under bone dry nitrogen and the membranes were carefully removed from the glass surface and cut into 4 mm² circles using a sterile skin biopsy punch device. PLGA membranes were stored at RT until use.

Collagen bilayers membranes were made by alkaline initiated polymerization of a Type I bovine collagen (Cell Prime, Collagen Biomaterials, Fremont, Calif.) solution using phosphate buffered saline, pH 7.2 (Life Technologies, Grand Island, N.Y.) as a diluent. The concentration of type I collagen in both layers of the biofilm was 2.2 mg/ml. The base layer of the biofilm was cast into the bottom of two well chamber slides (Nalge Nunc International, Naperville, Ill.) using a total volume of 1 ml per well. The base layer was polymerized for a period of 24 hours at 37° C. prior to the application of the top layer. The top layer consisted of a total volume of 500 μl collagen solution containing 5% phosphatidylglycerol (wt/wt %, Avanti Polar Lipids, Alabaster, Ala.). Membranes for in vitro transfections were prepared in a similar way with the exception that the membranes contained fibronectin-like peptides (1 mg/ml, Sigma). Membranes were allowed to polymerize and cure for a minimum of three days at 37° C. in a humidified atmosphere prior to the application of the PAMAM dendrimer DNA complexes.

For in vitro studies dendrimer/DNA complexes prepared in 25-50 μl of water at the indicated charge ratios were coated on the surface of the bilayer, or incorporated into the top layer during membrane polymerization. For in vivo studies, membranes were coated with 10-100 μg of pCF1CAT DNA alone or complexed with E5 EDA dendrimers at 1 and 5 dendrimer/DNA charge ratios. Dendrimer/DNA complexes were prepared in 100 μl of water and after incubation for 10 min at RT overlaid on the surface of the membranes. Coated membranes were air-dried in a laminar flow hood for 1-2 hr at RT prior to use.

### Example 3

Plasmids

The following reporter plasmids were employed in these studies: pCF1-Luc, pCF1 CAT and pEGF1. These plasmids have been described in detail elsewhere (See e.g., Yew et al., Human Gene. Ther., 8:575 [1997]; Raczka et al., Gene Ther 5:1333 [1998]; Baumann et al., J. Histochem. Cytochem., 46:1073 [1998]). Plasmid DNA was amplified in bacteria and then isolated by double cesium chloride gradient (See Tang et al., Biocong Chem 7:703 [1996]) to ensure the purity (e.g., removal of endotoxin) of the DNA preparation.

### Example 4

Preparation of Plasmid DNA/Dendrimer Complexes

Dendrimers were diluted to an appropriate concentration in water and all solutions were stored at 4° C. until required DNA/dendrimer complexes were formed by incubating the two components together in 100-200 μl of water for a minimum of 10 minutes at room temperature. Charge ratios of dendrimer to nucleic acid were based on the calculation of the electrostatic charge present on each component and the number of terminal NH₂ groups on the dendrimer versus the number of phosphate groups in the nucleic acid as previously described (See e.g., Kukowska-Latallo et al., Proc Natl Acad Sci USA 93:4897 [1996]; Bielinska et al., Nucleic Acids res 24:2176 [1996]; Bielinska et al., Biochim Biophs Acta 1353:180 [1997]; Bielinska et al., Bioconj Chem 10:843 [1999]).

### Examples 5

Cells and Media

This example describes the cell lines and media used in some of the embodiments of the present invention. COS-1, NIH 3T3 and Rat2 cells were maintained in D-MEM medium (Gibco BRL, Life Technologies, Rockville, Md.) with 5%-10% fetal calf serum (FCS) (Hyclone, Logan, Utah), 1% penicillin-streptomycin and 2 mM L-glutamine. NBF1 cells were cultivated in RPNI 1640 (Life Technologies, Rockville, Md.) supplemented with 10% fetal calf serum, 1% penicillin-streptomycin, 2 mM L-glutamine, 50 μM 2-mercaptoethanol, 1 mM non-essential amino acids. Primary human epithelial keratinocytes (PHEK) were purchased from Clonetics and grown in keratinocyte SFM medium (Clonetics, Walkersville, Md.). For transfection experiments cells were seeded and grown at the subconfluent densities 50-70%. All cell lines were incubated at 37° C. in 5% CO₂.

### Example 6

### In Vitro Transfection Method

This example describes in vitro dendrimer-based delivery experiments using the methods and compositions of the present invention. Transfection with dendrimer/plasmid DNA complexes (See e.g., Example 4) were performed and analyzed using assays for luciferase activity expression from pCF1-Luc and pEGFP1 reporter plasmids (See e.g., Yew et al., Human Gene. Ther., 8:575 [1997]; Raczka et al., Gene Ther 5:1333 [1998]; Baumann et al., J. Histochem. Cytochem., 46:1073 [1998]). Indicated amounts (μg) of pCF1-Luc DNA or pEGFP1 (coding green fluorescent protein) were mixed with dendrimers at a variety of dendrimer to DNA charge ratios (ranging from 1 to 50) in water and were allowed to form complexes for 5 to 10 minutes at RT. For standard solution-based transfections 24-well plates were seeded 24 hours before the transfection with approximately 2×10⁴ cells per well. The dendrimer/DNA complexes were added directly to serum free medium and transfection was carried on for 3 hours at 37° C. Following incubation with dendrimer/DNA complexes cells were washed with serum free medium and returned to complete growth media. The cells were harvested 24 or 48 hr following transfections and assayed for the expression of luciferase.

To analyze transfectional properties of Dendrimer/DNA complexes coated on the surface of, or incorporated into the collagen membranes, cells were seeded directly on the surface of the membranes in the presence of medium supplemented with 5% FBS and incubated for 3-4 hr followed by 24-48 or 72 hr incubation in the appropriate full growth medium. In all cases luciferase activity was determined by measuring the light emission from 10 μl of cell lysate incubated with 2.35×10⁻² μmoles of luciferin substrate (Promega, Technical Bulletin No.101). Light emission was measured in a chemiluminometer (LB96P, EG&G Berthold, Madison, Wis.), and adjusted to the protein concentration of the sample. The protein concentration in the cell lysates was measured in a standard protein assay (DC protein assay, Bio-Rad, Richmond, Calif.).

### Example 7

Collagenase Treatment

This example describes the treatment of collagen membranes with collagenase. Collagen membranes with dendrimer/DNA complexes incorporated into the film were incubated at 37° C. with 0.01 mg/ml collagenase (Sigma blend, #C8301) (Sigma, St. Louis, Mo.). After 30 min of incubation, the collagenase was removed and films continued to incubate for an additional 30 min. Later, approximately 5×10² cells/cm² were seeded in the full growth medium, and incubated 24 h before harvesting.

### Example 8

### Animals and In Vivo Transfections

This example describes in vivo dendrimer-based delivery experiments according to the methods and compositions of the present invention. Male hairless mice (Skh-hr⁻¹, 60 days old, Charles River Breeding Laboratories, Wilmington, Del.) were anesthetized with 30 mg/kg intraperitoneal injection of sodium pentobarbital. The flank skin of the animals was stripped using cellophane tape a total of 15 times. The membrane was then placed over the stripped area and the edges of the membrane were adhered to the skin using cyanoacrylate glue. The membrane was then covered using an occlusive dressing of petrolatum gauze and sterile gauze wraps to prevent removal of the membranes by the animal. The animals were then placed in individual cages for 24 hours or 48 hours at which time they were sacrificed and the skin processed as described below.

### Example 9

### Harvest of Skin for Transfection Assays

This example describes the harvest of skin from test animals following the administration dendrimer-based methods and compositions of the present invention. At 24 h or 48 h the animals were sacrificed with a lethal dose injection of sodium pentobarbital. The self-adherent wrap was then unwrapped and punch biopsies of skin, either 3 mm or 4 mm in diameter (Baker Biopsy Punch), were then obtained from the area exposed to treatment. A total of seven to ten biopsies were typically collected and placed in Eppendorf tubes. Generally, a maximum of four such biopsies were placed in one Eppendorf tube. The punch biopsies were snap frozen and stored at −70° C. until the extraction procedure was undertaken.

### Example 10

### Extraction of Protein from Skin Tissues of Hairless Mice

This example describes the extraction of protein from the skin of test animals following administration of the dendrimer-based methods and compositions of the present invention. 100 μl of 1% chloramphenicol acetyltransferase (CAT) lysis buffer (Boehringer Mannheim GmbH, Indianapolis, Ind.) was added to each tube containing the skin tissues and mixed by vortexing a few seconds. The tubes containing skin tissue were always kept on ice. A probe sonicator (Micro Ultrasonic Cell Disruptor, Kontes, Inc.) was used to homogenize the skin in each tube under the following conditions; 40 W and output: 60 (range from 0 to 100). The samples were sonicated two times and each time sonication consisted of 7 pulses. The interval between the two sonications was approximately 10 minutes. The samples were then centrifuged at 5,000 rpm and 4° C. for 20 minutes. The supernatants from each tube were then pooled and sonicated a total of six times using the conditions described above.

### Example 11

### Chloramphenicol Acetyltransferase ELISA

This example describes a method used to quantify chloramphenicol acetyltransferase CAT expression in skin target cells following administration of the methods and compositions of the present invention. 50-100 μl of skin homogenate was analyzed in CAT ELISA (Boeringer Manheim GmbH, Indianapolis, Ind.) per the manufacturers instructions. The amount of CAT protein was adjusted to the protein concentration of the samples.

### Example 12

### Histochemical Staining for Chloramphenicol Acetyltransferase Activity

This example describes a histochemical staining procedure used in the present invention. Skin biopsies (4 mm) were fixed, parafinized and serial sections (5 μm) were obtained. Histochemical staining of CAT activity in skin sections was performed using a CAT staining kit (Boeringer Manheim GmbH, Indianapolis, Ind.) according to the manufacture's recommended staining procedure. After 12-24 h incubation at RT, slides were rinsed in water and counterstained with hemotoxilin and eosin (H-O). Slides were mounted with 100 μl of GVA-mount, and photographed with an Olympus BH-2 microscope.

### Example 13

### Statistical Analysis Methods

This example describes the statistical analysis performed on results from the dendrimer-based delivery methods and compositions of the present invention. Statistical analysis was performed using Systat 5.2 software for Macintosh (Heame Scientific Software, Melbourne, Australia). Errors were calculated as standard deviations and differences between samples were analyzed by ANOVA.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in relevant fields, are intended to be within the scope of the following claims.

We claim:

1. A composition comprising: a membrane associated with at least one dendrimer, said dendrimer comprising at least one biological agent.

2. The composition of claim 1, wherein said membrane comprises a biocompatible membrane.

3. The composition of claim 1, wherein said membrane comprises a bioerodable membrane.

4. The composition of claim 1, wherein said membrane is desiccated.

5. The composition of claim 1, wherein said membrane comprises a PLGA membrane.

6. The composition of claim 1, wherein said membrane comprises a collagen membrane.

7. The composition of claim 1, wherein said dendrimer is covalently attached to said membrane.

8. The composition of claim 1, wherein said dendrimer is attached to a surface of said membrane.

9. The composition of claim 1, wherein said dendrimer is encompassed within said membrane.

10. The composition of claim 1, wherein said membrane is associated with a plurality of dendrimers.

11. The composition of claim 1, wherein said agent is attached to a surface of said dendrimer.

12. The composition of claim 1, wherein said agent is encompassed within said dendrimer.

13. The composition of claim 1, wherein said agent comprises a therapeutic agent.

14. The composition of claim 13, wherein said therapeutic agent comprises nucleic acid.

15. The composition of claim 14, wherein said nucleic acid comprises DNA.

16. The composition of claim 15, wherein said DNA comprises a gene encoding a protein that promotes wound healing.

17. The composition of claim 16, wherein said gene comprises a gene encoding a growth factor.

18. The composition of claim 15, wherein said DNA comprises a gene encoding a protein that promotes tissue vascularization.

19. The composition of claim 18, wherein said gene comprises a gene encoding a growth factor.

20. The composition of claim 13, wherein said therapeutic agent comprises a protein.

21. The composition of claim 20, wherein said protein comprises a protein that promotes wound healing.

22. The composition of claim 21, wherein said protein comprises a growth factor.

23. The composition of claim 20, wherein said protein comprises a protein that promotes tissue vascularization.

24. The composition of claim 23, wherein said protein comprises a growth factor.

25. A method comprising:
a) providing:
i) a tissue; and
ii) a composition comprising a membrane associated with at least one dendrimer, said dendrimer comprising at least one biological agent; and
b) contacting said tissue with said composition.

26. The method of claim 25, wherein said tissue comprises cultured cells in vitro.

27. The method of claim 25, wherein said tissue comprises skin cells.

28. The method of claim 25, wherein said tissue comprises ex vivo tissue obtained from a subject.

29. The method of claim 25, wherein said tissue comprises tissue of a subject.

30. The method of claim 29, wherein said contacting comprises placing said composition on a wound of said subject.

31. The method of claim 29, wherein said contacting comprises placing said composition on a lesion of said subject.

32. The method of claim 25, wherein said membrane comprises a biocompatible membrane.

33. The method of claim 25, wherein said membrane comprises a bioerodable membrane.

34. The method of claim 25, wherein said membrane is desiccated.

35. The method of claim 25, wherein said membrane comprises a PLGA membrane.

36. The method of claim 25, wherein said membrane comprises a collagen membrane.

37. The method of claim 25, wherein said dendrimer is covalently attached to said membrane.

38. The method of claim 25, wherein said dendrimer is attached to a surface of said membrane.

39. The method of claim 25, wherein said dendrimer is encompassed within said membrane.

40. The method of claim 25, wherein said membrane is associated with a plurality of dendrimers.

41. The method of claim 25, wherein said agent is attached to a surface of said dendrimer.

42. The method of claim 25, wherein said agent is encompassed within said dendrimer.

43. The method of claim 25, wherein said agent comprises a therapeutic agent.

44. The method of claim 43, wherein said therapeutic agent comprises nucleic acid.

45. The method of claim 44, wherein said nucleic acid comprises DNA.

46. The method of claim 45, wherein said DNA comprises a gene encoding a protein that promotes wound healing.

47. The method of claim 46, wherein said gene comprises a gene encoding a growth factor.

48. The method of claim 45, wherein said DNA comprises a gene encoding a protein that promotes tissue vascularization.

49. The method of claim 48, wherein said gene comprises a gene encoding a growth factor.

50. The method of claim 43, wherein said therapeutic agent comprises a protein.

51. The method of claim 50, wherein said protein comprises a protein that promotes wound healing.

52. The method of claim 51, wherein said protein comprises a growth factor.

53. The method of claim 50, wherein said protein comprises a protein that promotes tissue vascularization.

54. The method of claim 53, wherein said protein comprises a growth factor.

55. A composition comprising a desiccated membrane capable of transfecting a tissue.

56. The composition of claim 55, wherein said membrane comprises at least one dendrimer.

57. The composition of claim 55, wherein said dendrimer comprises at least on biological agent.

58. The composition of claim 55, wherein said biological agent comprises nucleic acid.

59. The composition of claim 55, wherein said tissue comprises skin tissue.

60. A method comprising:
a) providing:
i) a tissue; and
ii) composition comprising a desiccated membrane capable of transfecting said tissue; and
b) contacting said tissue with said composition.

61. The composition of claim 60, wherein said membrane comprises at least one dendrimer.

62. The composition of claim 60, wherein said dendrimer comprises at least on biological agent.

63. The composition of claim 60, wherein said biological agent comprises nucleic acid.

64. The composition of claim 60, wherein said tissue comprises skin tissue.
